(19) United States
(12) Patent Application Publication
(10) Pub. No.: US 2022/0028560 A1
(43) Pub. Date: Jan. 27, 2022

(54) Systems and Methods for Real-Time Bio-Risk Determination

(71) Applicant: Biosafety Diagnostics, Inc., Hoboken, NJ (US)

(72) Inventors: Michael Kotlarz, Short Hills, NJ (US); Keith Barksdale, Hoboken, NJ (US)

(73) Assignee: Biosafety Diagnostics, Inc., Hoboken, NJ (US)

(21) Appl. No.: 17/223,861

(22) Filed: Apr. 6, 2021

Related U.S. Application Data
(63) Continuation-in-part of application No. 17/037,563, filed on Sep. 29, 2020.
(60) Provisional application No. 63/055,201, filed on Jul. 22, 2020.

(51) Int. Cl. G16H 50/80; C12Q 1/686

(52) U.S. Cl. CPC G16H 50/80; C12Q 1/686

(57) ABSTRACT

A bio-risk determination system is provided. The system includes a database including information on an individual, the database configured to receive data relating to an activity the individual is to participate in at a future time at a location. The system includes a processing device configured to receive a first input at a user interface of a mobile electronic device from the individual in response to a first query during a first temporal period prior to the activity, and receive a second input at the user interface from the individual in response to a second query during a second temporal period prior to the activity. The processing device is configured to generate a bio-risk of the individual based on the first and second inputs in real-time, and display a report in the user interface of a mobile electronic device, the report indicating the bio-risk generated for the individual.

### RELATED APPLICATIONS

This application is a United States Non-Provisional patent application which claims priority to U.S. Non-Provisional application Ser. No. 17/037,563, filed on Sep. 29, 2020, which claims priority to U.S. Provisional Patent Application No. 63/055,201, filed on Jul. 22, 2020, the entire contents of the foregoing patent application are hereby expressly incorporated by reference.

### BACKGROUND

### Technical Field

The present disclosure relates generally to the field of computer-based health tracking and data systems. More specifically, the present disclosure relates to systems and methods for real-time health certification using point-of-care data. The present disclosure further relates generally to the field of computer-based, dynamic data systems and methods for real-time for bio-risk certification.

### Related Art

In today's world, real-time tracking of the health of individuals is gaining increased importance. Recently, global health pandemics (such as the current coronavirus pandemic) have made the detection and tracking of individuals who have tested positive for illnesses particularly important in assisting with curtailing the spread of such illnesses. Moreover, the ability to assess, in real time, the health risk posed by individuals is likely to be highly useful in connection with activities, events, transactions, and locations (both physical and virtual) where biological risk may be a concern.

Point-of-care health data represents an important source of information that can be used to ascertain the health risks of individuals. Examples of such data include, but are not limited to, manual and automated laboratory test results, medical treatment and/or diagnosis results, private testing results, and other sources of point-of-care health data. A particular technological problem with such point-of-care health data is that the information is often stored in proprietary, incompatible data formats. Moreover, currently there is not an acceptable real-time system that can communicate with disparate point-of-care health data systems, consolidate the data into a consolidated, centralized, real-time database, and processes the consolidated data rapidly in order to provide a real-time risk assessment of whether a particular individual suffers from a biological risk that may make participation in an activity, event, or transaction, or attendance at a particular location, inadvisable from a public health and safety perspective. For example, in the current coronavirus pandemic, what is lacking is a real-time system that can rapidly ascertain the risk level of a person who may be suffering from coronavirus using point-of-care data stored in various, disparate, and often incompatible data platforms formats, and which can be used to monitor and/or regulate such person's future activities. Still further, there is currently a need for a real-time system that can be utilized to electronically certify that a person is approved to engage in an activity and/or to be present at a location, using point-of-care data.

Accordingly, what would be desirable, but has not yet been provided, are systems and methods for real-time bio-risk determination using medical and/or non-medical data which solve the foregoing and other needs.

### SUMMARY

This present disclosure relates to systems and methods for real-time health certification using point-of-care data. The system includes a consolidated, centralized, real-time database that stores point-of-care data in a common storage format, and a certification server in communication with the database. The point-of-care data could include, but is not limited to, manual and automatic laboratory test results, medical (e.g., hospital) patient information, private test results, and mandated test results. The certification server processes the real-time database and calculates a risk score for an individual, and can generate an electronic report for the patient in response to an electronic query, which includes the risk score for the patient, an indication of whether the individual is currently suffering from an illness, and optionally, a data symbol (e.g., a QR code) which electronically encodes the report and can be used to electronically certify that the individual is authorized to engage in one or more events or activities, access one or more services or devices, and/or attend one or more locations. Optionally, the report can be accessed by computer systems maintained by one or more third-parties such as corporate entities, travel entities, electronic medical records custodians, educational entities, healthcare entities, medical research entities, governmental entities, and/or other third-party entities, using one or more customized application programming interfaces (APIs).

The present disclosure relates to a health pass system for COVID-19. The system includes a database including information on a plurality of individuals. The database is configured to receive point-of-care data relating to the plurality of individuals. The point-of-care data includes at least one medical test result for COVID-19 antibodies for at least one of the plurality of individuals. The system includes a processing device configured to analyze the point-of-care data and generate a report. The system includes means for transmitting the report to a mobile electronic device of at least one of the plurality of individuals. The system includes means for displaying the report with a user interface of the mobile electronic device in the form of a real-time health certification. The report includes the at least one medical test result for COVID-19 antibodies for the at least one individual, and an indication of a positive or negative result of the at least one medical test result for COVID-19 antibodies. The report can include a code (e.g., a QR code, a bar code, or the like) capable of being scanned to display information associated with the report on another user interface or an electronic device.

In some embodiments, the report can include a photograph of the individual. In some embodiments, the report can include a unique alphanumerical identification associated with the individual. In some embodiments, the unique alphanumerical identification can be used to access the report. In some embodiments, the report can include, e.g., a name of the individual, a type of test performed for the at least one medical test result, a date of the test performed for the at least one medical test result, combinations thereof, or the like. The point-of-care data can include at least one of manual laboratory test results, automatic laboratory test results, medical information, private test results, or mandated test results. The point-of-care data can include information relating to or derived from at least one of DNA, antibodies, psychological evaluation, existing conditions, medical history, plasma, blood composition, saliva, bodily fluid, stool, bone marrow, enzymes, glucose, cholesterol, immunology, gastric fluid, kidney function, inulin, pregnancy, toxicology, urinalysis, thyroid function, genetic testing, or skin testing.

The at least one medical test result for COVID-19 antibodies can include results for IGG+ and IGM− antibodies. In some embodiments, the database can be configured to receive the point-of-care data from medical tests in real-time. In some embodiments, the database can be configured to receive the point-of-care data from already administered medical tests. In some embodiments, the database can be configured to receive the point-of-care data from an automated testing system or a photograph of a medical test result received as input.

The processing device can be configured to consolidate the point-of-care data and convert the point-of-care data into a common storage format having a unified data structure. The processing device can be configured to generate a risk score based on analysis of the point-of-care data. The risk score can indicate one or more biological risks associated with the plurality of individuals. In some embodiments, the processing device can be configured to generate the risk score based on the point-of-care data and medical guidelines including thresholds for the point-of-care data regarding biological illness levels or risks. In such embodiments, the processing device can be configured to automatically update the risk score based on real-time updated medical guidelines.

In some embodiments, the processing device can be configured to generate the risk score based on the point-of-care data, prior conditions, demographics, and data collected at a point of presence. The report can include the risk score. In some embodiments, based on the generated risk score, the report can include a designation of safe or note safe for the at least one of the plurality of individuals (as an alternative to or in combination with the risk score).

The present disclosure relates to a method of generating a health pass for COVID-19. The method includes receiving at a database information a plurality of individuals. The method includes receiving at the database point-of-care data relating to the plurality of individuals. The point-of-care data includes at least one medical test result for COVID-19 antibodies for at least one of the plurality of individuals. The method includes analyzing the point-of-care data and generating a report with a processing device. The method includes transmitting the report to a mobile electronic device of at least one of the plurality of individuals. The method includes displaying the report with a user interface of the mobile electronic device in the form of a real-time health certification. The report includes the at least one medical test result for COVID-19 antibodies for the at least one individual, an indication of a positive or negative result of the at least one medical test result for COVID-19 antibodies, and a code (e.g., a QR code, a bar code, or the like) capable of being scanned to display information associated with the report on another user interface or an electronic device.

In some embodiments, the report can include, e.g., a photograph of the individual, a name of the individual, a type of test performed for the at least one medical test result, a date of the test performed for the at least one medical test result, combinations thereof, or the like. In some embodiments, the at least one medical test result for COVID-19 antibodies includes results for IGG+ and IGM− antibodies.

The present disclosure relates to non-transitory computer-readable medium storing instructions at least for generating a health pass for COVID-19 that are executable by a processing device. Execution of the instructions by the processing device causes the processing device to receive at a database information a plurality of individuals. Execution of the instructions by the processing device causes the processing device to receive at the database point-of-care data relating to the plurality of individuals. The point-of-care data includes at least one medical test result for COVID-19 antibodies for at least one of the plurality of individuals. Execution of the instructions by the processing device causes the processing device to analyze the point-of-care data and generate a report. Execution of the instructions by the processing device causes the processing device to transmit the report to a mobile electronic device of at least one of the plurality of individuals. Execution of the instructions by the processing device causes the processing device to display the report with a user interface of the mobile electronic device in the form of a real-time health certification. The report includes the at least one medical test result for COVID-19 antibodies for the at least one individual, an indication of a positive or negative result of the at least one medical test result for COVID-19 antibodies, and a code (e.g., a QR code, a bar code, or the like) capable of being scanned to display information associated with the report on another user interface or an electronic device. In some embodiments, the report can include, e.g., a photograph of the individual, a name of the individual, a type of test performed for the at least one medical test result, a date of the test performed for the at least one medical test result, combinations thereof, or the like.

The present disclosure relates to bio-risk determination systems. The systems include a database including information on an individual. The database is configured to receive data relating to an activity in which the individual is to participate at a future time at a location. The system includes a processing device configured to receive a first input at a user interface of a mobile electronic device from the individual in response to a first query during a first temporal period prior to the activity. The processing device is configured to receive a second input at the user interface from the individual in response to a second query during a second temporal period prior to the activity. The second temporal period is different from the first temporal period. The processing device is configured to analyze the first and second inputs, and generate a bio-risk of the individual based on the first and second inputs in real-time. The processing device is configured to display a report in the user interface of a mobile electronic device. The report indicates the bio-risk generated for the individual.

The activity can include (but is not limited to) at least one of a sports event, a musical event, airline travel, bus travel, or train travel. In some embodiments, the first query can be a request for a first medical test result associated with the individual. In some embodiments, the first medical test result can be based on a polymerase chain reaction (PCR) test. In some embodiments, the first medical test result can be based on a first medical test conducted remotely from the location of the activity (e.g., in a different facility or laboratory physically separated from the location of the activity). The first medical test result can be automatically input in real-time into the database from a medical facility conducting the first medical test.

In some embodiments, the second query can be an electronic questionnaire regarding symptoms of the individual (e.g., a symptoms survey). In some embodiments, the processing device can be configured to receive a third input at a user interface from the individual in response to a third query during a third temporal period prior to the activity. In some embodiments, the third query is a request for a second medical test result associated with the individual. In some embodiments, the second medical test result can be based on a COVID-19 antibody test. In some embodiments, the second medical test result can be based on a second medical test conducted at the location of the activity (e.g., a laboratory or medical facility associated with the location of the activity).

The third temporal period is different from the first and second temporal periods. The first temporal period is a longer time period from the activity than the second and third temporal periods, and the second temporal period is a longer time period from the activity than the third temporal period. In some embodiments, the second medical test can be conducted on the day on which the individual is participating in the activity (e.g., within an hour or a few hours of the activity start time).

The processing device can be configured to update the bio-risk of the individual based on the third input in real-time. In some embodiments, the report can include a code capable of being scanned to display information associated with the report on another user interface of an electronic device. In some embodiments, the code can be a QR code or a barcode. In some embodiments, the processing device can be configured to automatically update the bio-risk of the individual based on updates in medical or government guidelines or thresholds regarding a biological illness level or risk. In some embodiments, the database can be configured to receive as input and electronically store data relating to an environment associated with the individual.

In some embodiments, the processing device can be configured to analyze a dynamic hot spot map (e.g., a hot spot map generated by the system, a hot spot map externally generated based on public information, combinations thereof, or the like) based on the data relating to the environment associated with the individual. In some embodiments, the processing device can be configured to automatically update the bio-risk associated with the individual based on travel of the individual through hot spots of the dynamic hot spot map.

The present disclosure relates to a method of determining a bio-risk. The method includes receiving at a database information on an individual, and receiving at the database data relating to an activity the individual is to participate in at a future time at a location. The method includes receiving a first input at a user interface of a mobile electronic device from the individual in response to a first query during a first temporal period prior to the activity. The method includes receiving a second input at the user interface from the individual in response to a second query during a second temporal period prior to the activity. The second temporal period is different from the first temporal period. The method includes analyzing the first and second inputs with a processing device, and generating a bio-risk of the individual based on the first and second inputs in real-time. The method includes displaying a report in the user interface, the report indicating the bio-risk generated for the individual.

The present disclosure relates to a non-transitory computer-readable medium storing instructions at least for generating a bio-risk that are executable by a processing device. Execution of the instructions by the processing device causes the processing device to receive at a database information on an individual, receive at the database data relating to an activity the individual is to participate in at a future time at a location. Execution of the instructions by the processing device causes the processing device to receive a first input at a user interface of a mobile electronic device from the individual in response to a first query during a first temporal period prior to the activity. Execution of the instructions by the processing device causes the processing device to receive a second input at the user interface from the individual in response to a second query during a second temporal period prior to the activity. The second temporal period is different from the first temporal period. Execution of the instructions by the processing device causes the processing device to analyze the first and second inputs, and generating a bio-risk of the individual based on the first and second inputs in real-time. Execution of the instructions by the processing device causes the processing device to display a report in the user interface, the report indicating the bio-risk generated for the individual.

The present disclosure relates to a bio-risk system for public transportation. The system includes a database including information on a plurality of individuals. The database is configured to receive data relating to a travel information (e.g., travel itinerary, ticket information, combinations thereof, or the like) associated with each of the plurality of individuals, and at least one medical test result associated with each of the plurality of individuals. The system includes a processing device configured to analyze the travel information and the at least one medical test result, and generate an electronic report. The processing device is configured to transmit the electronic report to a mobile electronic device of at least one of the plurality of individuals. The processing device is configured to display the report with a user interface of the mobile electronic device in the form of a real-time health certification, the report including at least one of the medical test result, or an indication of a positive or negative result of the medical test result. In some embodiments, the at least one medical test result is a COVID-19 antibody test result.

### BRIEF DESCRIPTION OF THE DRAWINGS

The foregoing features of the invention will be apparent from the following Detailed Description of the Invention, taken in connection with the accompanying drawings, in which:

FIG. 1 is a diagram illustrating hardware and software components of the system of the present disclosure;

FIG. 2 is a diagram illustrating the system of FIG. 1 in greater detail;

FIG. 3 is a diagram illustrating an example of an electronic health certification report capable of being generated by the system of the present disclosure;

FIG. 4 is a diagram illustrating another example of an electronic health certification report capable of being generated by the system of the present disclosure;

FIG. 5 is a diagram illustrating a visualization tool capable of being generated by the system of the present disclosure, for graphically depicting the locations of individuals at a venue;

FIG. 6 is a flowchart illustrating processing steps carried out by the system for calculating a health risk score for an individual;

FIG. 7 is a diagram illustrating hardware and software components of a bio-risk determination system of the present disclosure;

FIG. 8 is a sample screen shot of a user interface illustrating an example of an electronic certification report capable of being generated by the bio-risk determination system of the present disclosure;

FIG. 9 is a sample screen shot of a user interface illustrating an example of a polymerase chain reaction (PCR) test input capable of being generated by the bio-risk determination system of the present disclosure;

FIG. 10 is a sample screen shot of a user interface illustrating an example of PCR administration instructions capable of being generated by the bio-risk determination system of the present disclosure;

FIG. 11 is a sample screen shot of a user interface illustrating an example of a PCR acceptance dashboard capable of being generated by the bio-risk determination system of the present disclosure;

FIG. 12 is a sample screen shot of a user interface illustrating an example of a clearance protocols status capable of being generated by the bio-risk determination system of the present disclosure;

FIG. 13 is a sample screen shot of a user interface illustrating an example of a symptoms survey input capable of being generated by the bio-risk determination system of the present disclosure;

FIG. 14 is a sample screen shot of a user interface illustrating an example of an antigen clearance protocol capable of being generated by the bio-risk determination system of the present disclosure;

FIG. 15 is a sample screen shot of a user interface illustrating an example of a laboratory check-in dashboard capable of being generated by the bio-risk determination system of the present disclosure;

FIG. 16 is a sample screen shot of a user interface illustrating an example of an antigen results dashboard capable of being generated by the bio-risk determination system of the present disclosure;

FIG. 17 is a sample screen shot of a user interface illustrating an example of an electronic certification report capable of being generated by the bio-risk determination system of the present disclosure;

FIG. 18 is a sample screen shot of a hot spots map capable of being generated by the bio-risk determination system of the present disclosure.

FIG. 19 is a diagram illustrating an example of a computing device capable of being implemented with the bio-risk determination system of the present disclosure;

FIG. 20 is a diagram illustrating an example bio-risk determination system environment of the present disclosure; and

FIG. 21 is a flowchart illustrating an example implementation of the bio-risk determination system of the present disclosure.

### DETAILED DESCRIPTION

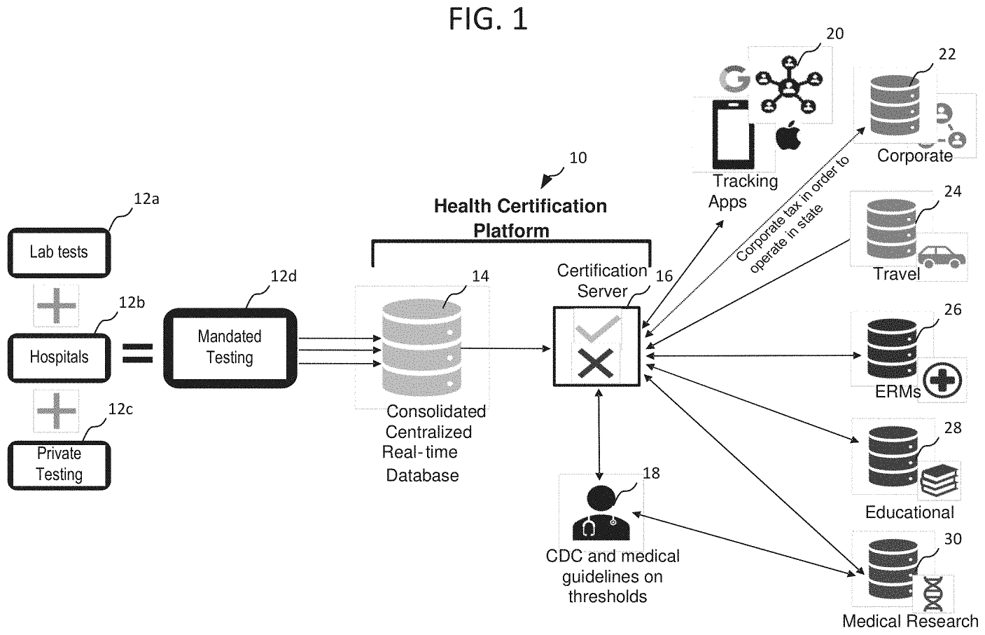

The present disclosure relates to systems and methods for real-time health certification using point-of-care data, as described in detail below in connection with FIGS. 1-6.

FIG. 1 is a diagram illustrating hardware and software components of the health certification system 10 (hereinafter “system 10”) of the present disclosure. Components 12a-12d of the system 10 represent a “testing” phase, components 14-18 of the system 10 represent a “tracking” phase, and components 20-30 represent a “tracing” phase, as will be described in greater detail below. In the “testing” phase (e.g., a collection phase), point-of-care data from a variety of sources can be collected in real-time. The point-of-care data could include, but is not limited to, manual and automatic laboratory test results (e.g., lab tests 12a), medical (e.g., hospital) patient information (e.g., hospital testing 12b), private test results (e.g., private testing 12c), and mandated test results (e.g., mandated testing 12d).

The point-of-care data can be collected from a test in real-time, and/or from an already administered test. As an example the lab tests 12a, hospital testing 12b and private testing 12c can be used to collect data on a variety of individuals, e.g., first responders, teachers, students, employees, citizens, combinations thereof, or the like. As a further example, the mandated testing 12d can be used to collect data focused on specific biological illnesses or infectious diseases. Such mandated testing 12d can include, but is not limited to, COVID-19, SARS, MERS, measles, or the like. The types of biological information that can be used for collection of the point-of-care data can include, but is not limited to, DNA, antibodies, psychological evaluation data, existing conditions, medical history, plasma, blood composition, plasma, saliva, any bodily fluid, urine, stool, bone marrow, enzymes, glucose, cholesterol, immunology, gastric fluid, kidney function, inulin, pregnancy, toxicology, urinalysis, thyroid function, genetic testing, skin testing, or the like.

The point-of-care data can be collected through, e.g., manual input into the system 10, wireless computer protocols, LIS servers, HL7 diagnostic protocols, HIPAA compliant database queries, batch processing from employment and medical records, insurer records, any other means of digital entry, or the like. In some embodiments, the system 10 can use machine vision to read and analyze the results of manual tests given the make and model of the test, and populates the data into the system 10. For example, if a cell phone photograph is received as input by the system 10 for a manual test, the system 10 can use machine vision to retrieve details of the point-of-care data from the test for further analysis. The system 10 can also read or retrieve the point-of-care information directly from an automated testing system.

In some embodiments, each individual for whom data is collected can complete a registration form for consent of participation in the system 10. Such registration can result in a unique alphanumerical identification (e.g., a personal or person ID) associated with the specific individual for collecting and maintaining point-of-care data for the individual over time. The system 10 can aggregate the data from the “testing” phase into a consolidated centralized real-time database 14 from all testing locations. In aggregating the data, the system 10 can convert the point-of-care data into a common storage format to ensure all data can be used effectively to evaluate the biological risk of the individual associated with each person ID. In particular, the system 10 can aggregate various machine or lab results to centralize all test results for the individual, and consolidates and translates various datatypes and formats into unified data structure. The system 10 can thereby operate and integrate with any testing system. In some embodiments, the system 10 can process 1,000,000 or more data points/second.

A certification server 16 can receive as input the consolidated point-of-care data from the database 14, and generates a risk score for each individual based on the point-of-care data. The generated risk score can also be tied to the person ID based on the person ID used for the consolidated point-of-care data. The system 10 can receive as input at the certification server 16 up-to-date guidelines 18 in real-time to assist in generation of the risk score. Such guidelines 18 can include, e.g., CDC and/or medical guidelines on thresholds for point-of-care data to evaluate the biological illness level or risk of an individual. The guidelines 18 can be updated in real-time as a set of rules for the risk score generation based on the ongoing and changing scientific understanding of the biological risk involved. Operation of the certification server 16 is thereby dynamic based on ongoing changed in the guidelines 18, as well as updated testing for the individual received at the database 14 in real-time.

In addition to using the testing data to evaluate and determine the risk score for the individual, the system 10 can receive as input an aggregate from other systems to assist in the analysis of the biological risk. The additional data can include, e.g., prior conditions, demographics, data collected at the point of presence, combinations thereof, or the like, and can be used to assist in rendering a risk score or rating. The risk scores can be output by the system 10 as safety levels, e.g., with level 1 being the safest, and each increasing level representing an additional level of risk associated with the individual.

For example, with respect to COVID-19, the certification server 16 can used antibody testing data for the analysis, as well as supplemental factors. In one instance, the certification server 16 analysis can be as follows: COVID-19 IGG+ with IGG index>20° and no prior conditions, IGM negative with index<0.5, body temperature<100° F., to output a safety level 1 or safest level for the individual. In another instance, the certification server 16 analysis can be as follows: COVID-19 IGG+ with IGG index>20 and IGG<0.80 and no prior conditions, IGM negative with index<1, body temperature<99° F., to output a safety level of 2 or the next safest level for the individual. The safety level association with the person ID can be automatically updated in real-time based on additional testing data received by the system 10 for the individual and/or based on updated guidelines 18 regarding biological risks associated with the individual. It should be understood that the certification server 16 analysis can include several other levels of supplemental data based on different inputs for determination of the risk score.

As a further example, with COVID-19, the system 10 can be initially programmed to a very high level of IGG antibodies (IGG+) and a very low level of IGM Antibodies (IGM−) to denote a low COVID-19 risk level, and borderline immunity. Such antibody levels can be used as thresholds for analysis by the certification server 16. As medical or governmental regulatory standards change (i.e., guidelines 18) and as the system 10 learns more about the disease through the updated guidelines 18, the thresholds of risk levels can be adjusted and applied to the existing dataset. The system 10 can thereby continuously or substantially continuously update the risk score for individuals based on the updated guidelines 18 based on testing data previously received by the system 10, reducing the need to re-test individuals if the data is available. In some embodiments, such updating by the system 10 can be based on the type of test taken previously by the individual. For example, in some instances, a re-test may be necessary to determine the current level of antibodies of the individual. The continuously or substantially continuously updating of the testing data can be similarly used for any biological data being recorded and aggregated, including other non-biological inputs that may inform the risk score determination. For example, non-biologic inputs that may affect the risk score determination can include place of origin and/or demographics where a known biologic risk is higher.

The risk score generated for each individual can be electronically stored in the certification server 16. In some embodiments, a separate biological risk level database can receive as input the risk score generated by the certification server 16, and the risk levels or risk scores electronically stored in the risk level database can be automatically updated in real-time based on continued analysis of the certification server 16. The risk level database can thereby be created using the biological factor raw data inputs and the logic used by the certification server 16 to create and calculate the risk level. The person ID number for each respective individual can be electronically tied to their corresponding calculated risk score. The person ID can be used as a unique electronic element or identifier to access or transact physically or electronically with subsequent queries for the risk score of the individuals, such as queries by physical/virtual venues, physical/virtual services, places of business, transit, education, commerce of any type, combinations thereof, or the like. The risk score and any data associated with the individual can be securely encrypted and authenticated in the respective database(s).

In some embodiments, the individual associated with the encrypted unique person ID number can be provided with a selection to allow the entity/user to electronically decide who has access to the risk score information and for what purpose. For example, the individual can selectively choose who has access to the risk score and associated information on a case-by-case basis. In some embodiments, the individual can provide a one-time confirmation to allow all future access to the risk score data until the individual decides to manually opt out of such access.

The score level and, optionally, additional data associated with the risk score determination, can be provided to a variety of requestors to gauge the risk level of the individual. As non-limiting examples, FIG. 1 depicts such requestors as the following industries or entities: tracking applications 20, corporate 22, travel 24, enterprise risk management (ERMs) 26, educational 28, medical research 30, combinations thereof, or the like. One or more customized application programming interfaces (APIs) can be used for retrieval and/or transmission of the data associated with the risk score based on queries from one or more individuals and/or entities. In some embodiments, each risk score can be retrieved in real-time, encrypted, with time-stamped verification, through a series of levels of detail appropriate to the authenticated requested party. An API with any customizable set of information interested to the authenticated requestor can be provided, depending on the requestor. For example, a request by an employer or a university may have more detail, such as the risk score and additional data that indicates the reasons for the particular risk score. As a further example, a request from an e-commerce entity can be merely “safe” or “not safe” without providing a specific risk score or detail about the risk score determination.

Access and transmission of the risk score to the endpoints requesting the information can be performed in a highly secure and dynamic manner. Each endpoint can be authorized by the individual to gain access to the appropriate API level of information according to the need of the requestor. Such authorization can be provided on a case-by-case basis (e.g., with the individual receiving a notification upon each request with an option to permit or deny access), or can be provided by the individual during registration for the system 10. Examples of automated decisions where requests for the risk score are initiated can include, e.g., allowing a transaction to occur, opening a door, or accessing a space. In such instances, prior to allowing the transaction, opening the door, or permitting access to a space, the endpoint can automatically request the risk score status of the individual and, if the risk score is below a predetermined threshold (indicating that the individual is safe), the endpoint can permit the desired action to occur. If the risk score is above a predetermined threshold (indicating that the individual is not safe), the desired action can be denied and a message can be displayed to the individual indicating the reason for such denial.

For example, an individual can order an UBER® vehicle and, before UBER® provisions a vehicle, UBER® can query the system 10 to determine the biological risk score of the potential passenger. If the risk score is above a predetermined threshold, UBER® can alert the individual that a vehicle cannot be provided. If the risk score is below a predetermined threshold, UBER® can proceed with providing a vehicle to the individual and, optionally, can indicate to the individual that the risk score is acceptable.

A similar process can be performed for airline tickets, sports tickets, restaurant reservations, or any number of situations where contact and crowds could pose a risk. In the travel industry, areas involving airline ticketing, boarding pass registration, passport/visa control, train ticketing, ticket scanning, or the like, can incorporate the system 10 to obtain the risk score of individuals and, based on the risk scores, determine whether the individual should be permitted in the respective areas or on the vehicle for transport. Similarly, transit areas (such as bus ticketing, bus access, subway ticketing, and subway access) can determine the score level of individuals prior to permitting admission.

Sports venues can query for risk scores for individuals prior to permitting ticket purchase and again prior to stadium/venue entry based on depiction or association of a person ID with the ticket. The employment industry can integrate the risk score determination by the system 10 with employee timeclock systems to check the employee's risk score each time a clock-in procedure or entry into an establishment occurs. The education industry can integrate the risk score determination by the system 10 with students and faculty management systems such that schools, colleges and universities can better meet the specific treatment and educational needs of their students. For example, a student who is considered not yet safe based on the associated risk score may be offered virtual education in place of in person education until the student is certified as safe by the system 10. As a further example, a faculty member who is considered not safe based on the associated risk score may be asked to remain off the educational facility premises until the faculty member is certified as safe by the system 10. Physical entry of individuals and/or vehicles into a location having any existing gate and/or security infrastructure can also incorporate the system 10 to determine the biological safety level of the individual prior to permitting entry. The system 10 can be integrated into Enterprise Resource Planning (ERP) and/or point-of-sale (POS) systems. With such integration, companies such as SQUARE®, who offer fully integrated connected POS systems, can offer the ability to make their small business customers and their clients safe.

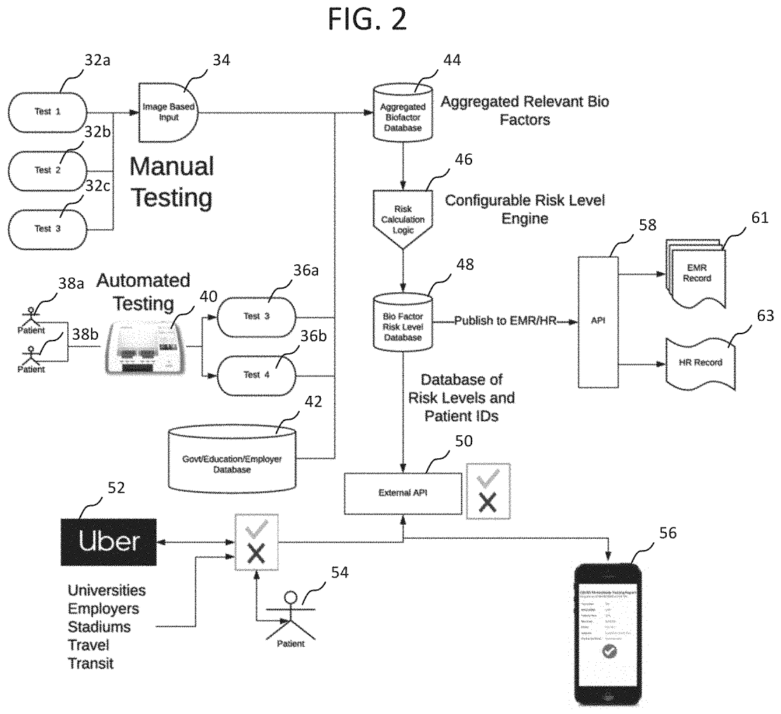

FIG. 2 is a diagram illustrating the system 10 of FIG. 1 in greater detail. As noted above, a variety of manual and/or automated testing can transmit point-of-care data to the system 10. Such data can be transmitted to the system 10 in real-time to ensure the most up-to-date information is being analyzed by the system 10. However, the system 10 can also receive as input point-of-care data for testing performed in the past, and can take into account the older point-of-care data in the risk score analysis. For manual testing, any number of tests 32a, 32b, 32c can be performed, and images 34 of the test results can be used as input into the system 10. The system 10 can in turn analyze the images 34 to determine the relevant data for extraction and further analysis. Automated testing can be performed for any number of patients 38a, 38b via an automated testing machine 40. For example, the automated testing machine 40 can be a finger prick machine to test for antibodies of the individual. In some embodiments, the finger prick machine can be a small and portable unit that provides results in substantially real-time (e.g., about 10 minutes). Automated results 36a, 36b of the tests can be output by the testing machine 40 and can be received as input by the system 10 for further analysis.

The point-of-care data can be electronically transmitted to an aggregated biofactor database 44 (e.g., database 14) that consolidates the data in a standard or uniform format capable of being used by the system 10. A database 42 maintaining updated medical guidelines for a variety of industries (e.g., government, education, employer, or the like) can be coupled to the system 10 and can be updated in real-time. A configurable risk level engine 46 (partially or fully incorporated in the certification server 16) having risk calculation logic programmed therein can receive as input the point-of-care data and the guidelines from database 42, and determines the risk, and/or risk score associated with each individual. The risk scores can be electronically stored in a biofactor risk level database 48, with each risk score associated with a unique person ID.

In some embodiments, the database 48 can be communicatively coupled to an internal API 58 for transmission of risk scores to EMR records 61 and human resources (HR) records 63. In some embodiments, external APIs 50 can be communicatively coupled to the database 48 to query the database 48 for risk scores associated with individuals. For external APIs 50, the system 10 can output the necessary information based on the type of entity requesting the information. For example, the system 10 can output to a requestor 52 an indication that the individual is safe or not safe. As a further example, the system 10 can output to a requestor via a graphical representation or report on an electronic device 56 a report with the risk score, whether the individual is safe or not safe, and additional data used for determining the risk status of the individual. As a further example, the system 10 can output to the patient 54 their risk status level.

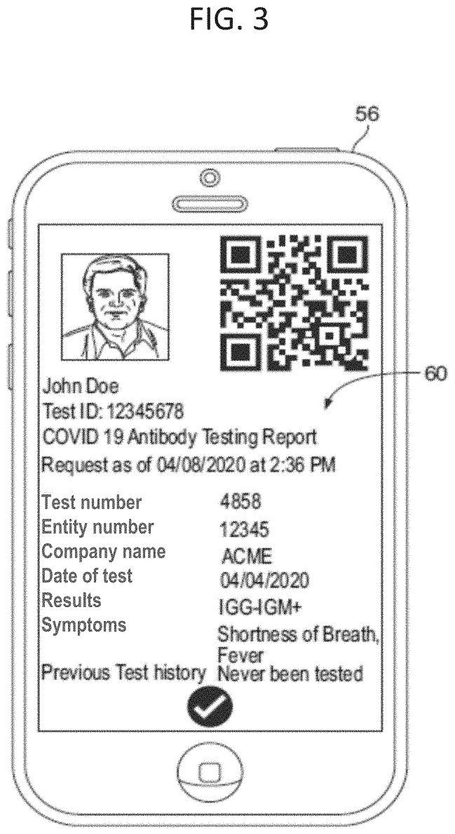

FIG. 3 is a diagram illustrating an electronic health certification report 60 capable of being generated by the system 10 of the present disclosure. The report 60 can be generated and depicted on an electronic device 56 (e.g., a mobile electronic device, a computer, or the like). The report 60 can also be provided in a standard format that can be forwarded or shared with additional parties. The report 60 can include, e.g., a photograph of the individual, the name of the individual, a person or test ID associated with the individual, a description of the type of biologic test performed, a date of request of the report, a test reference number, a requestor entity number, a requestor company name, a date of the biologic test, results of the biologic test, any symptoms felt by the individual, previous biologic test history of the individual, combinations thereof, or the like. In some embodiments, the report 60 can include a quick response (QR) code or barcode that can be scanned to transmit the report 60 to another individual. The report 60 of FIG. 3 indicates that the individual had a COVID-19 antibody test on Apr. 4, 2020, with results for IGG+ and IGM− antibodies, with the individual being asymptomatic at the time of the test, and no previous testing.

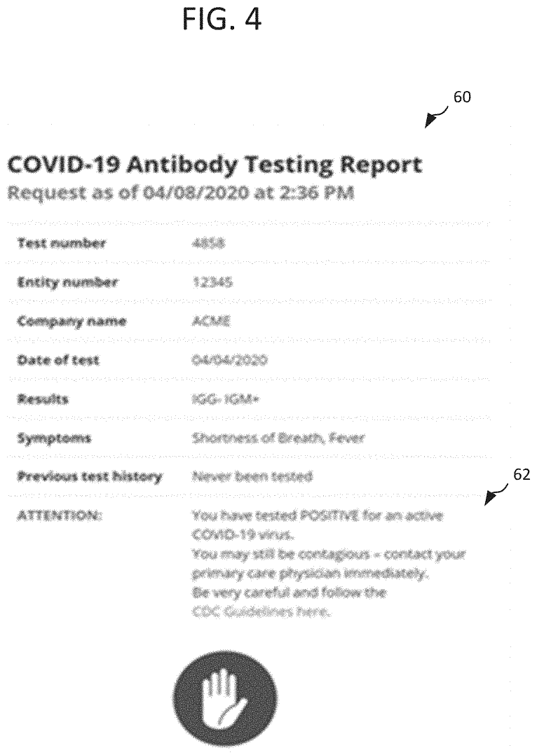

FIG. 4 is a diagram illustrating another electronic health certification report 60 capable of being generated by the system 10 of the present disclosure. The report 60 of FIG. 4 indicates that a COVID-19 antibody test was performed on Apr. 4, 2020, with results for IGG+ and IGM− antibodies. The report 60 further indicates that the patient had shortness of breath and a fever at the time of the test. If the system 10 determines that the individual may be a risk to others based on the tests performed, the report 60 can include an alert or attention section 62 in which important information can be presented. For example, the report 60 of FIG. 4 provides that the patient tested positive for the COVID-19 virus and may still be contagious in the attention section 62. In some instances, the graphic at the bottom of the report 60 can visually indicate whether the individual can proceed with the desired activity (e.g., purchasing a ticket, checking in to a flight, boarding a bus, or the like). In some instances, the attention section 62 can provide information about whether the individual can proceed with the desired activity.

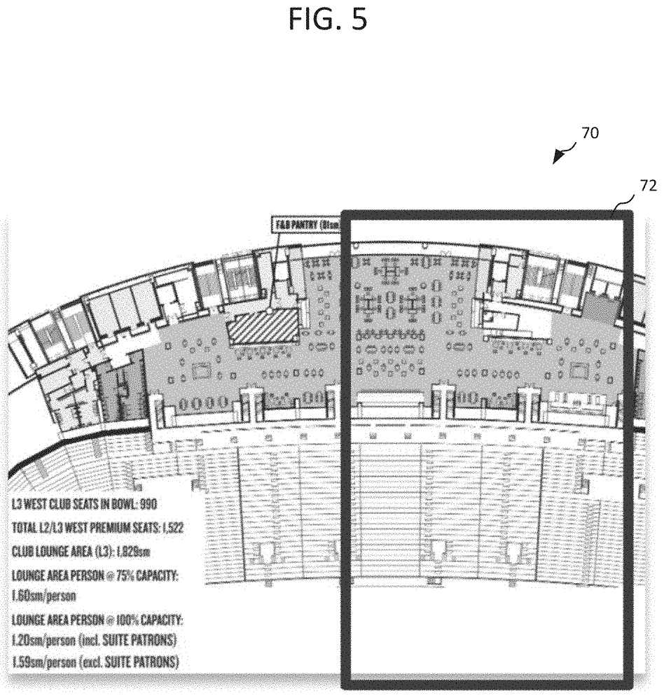

FIG. 5 is a diagram illustrating a visualization tool capable of being generated by the system 10 of the present disclosure, for graphically depicting the locations of individuals at a venue. As an example, FIG. 5 illustrates a partial map 70 of a seating arrangement in a stadium. The system 10 can provide means for selecting a specific section 72 of the interactive map 70 to obtain risk score or level information of individuals associated with the specific section 72. A venue operator can thereby select specific areas of focus on the map to determine the number of individuals in each area and whether one or more of the individuals create a risk to others in the area.

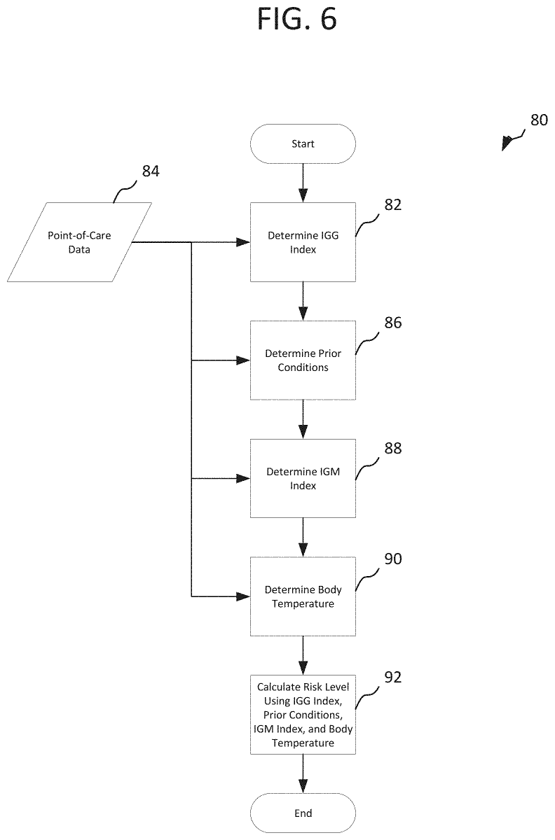

FIG. 6 is a flowchart illustrating processing steps 80 carried out by the system 10 for calculating a health risk of an individual, or a score for an individual. At step 82, the system 10 can determine the IGG antibody index for the individual, at step 86 the system 10 can determine any prior conditions associated with the individual, at step 88 the system 10 can determine an IGM antibody index for the individual, and at step 90 the system 10 determine the body temperature for the individual. Each of steps 82, 86, 88, 90 can receive as input point-of-care data collected at step 84 by the system 10. The point-of-care data can include manual testing and/or automated testing results, both in real-time and previously performed tests. Steps 82, 88 can incorporate medical guidelines associated with biological risks to determine whether the IGG index or the IGM index, respectively, are at levels below or above potential biological risks for the individual. At step 92, the IGG index, prior conditions, IGM index, and body temperature associated with the individual can be used to generate a risk score or level. The risk score or level can be updated at step 92 in a real-time or substantially real-time manner as additional point-of-care data is obtained and/or as medical guidelines are updated.

The system discussed herein therefore provides means for detecting and assigning a risk level for each individual tested based on the point-of-care data, and provides a real-time risk assessment of whether a particular individual suffers from a biological risk that may make participation in an activity, event, or transaction, or attendance at a particular location, inadvisable from a public health and safety perspective. Individuals with low risk levels can be permitted to proceed with participation in the activity, event, or transaction, or attendance at a particular location with minimal risks to the surrounding individuals. Individuals with high risk levels can be prevented from participation in the activity, event, or transaction, or attendance at the particular location to minimize risks to surrounding individuals. Individuals with low risk levels can thereby be permitted to work in safer environments, while ensuring point-of-care data is maintained in an encrypted manner to protect the individual.

The point-of-care data and risk levels can be updated in real-time based on additional testing and updates in medical guidelines. The system can collect data from a variety of sources, allowing for certification of individuals at any global location in real-time. The enrollment process of individuals can also be efficient, providing individuals with an opportunity to decide which entities are permitted to access the risk score associated with the individual and any additional data associated with the risk score.

The testing components of the system can receive point-of-care data from manual and/or automated testing locations, including mandatory testing at approved locations. Such locations can provide testing results indicating the biological levels associated with a variety of biological risks, such as antibodies associated with COVID-19. The tracking components of the system can populate one or more databases with the point-of-care data and medical guidelines, with such data and guidelines used by the system to generate a risk score or level for each individual. Such risk score or level can be used for first responders, students, employees, citizens, combinations thereof, or the like, to determine if such individuals can participate in certain events in environments having other individuals. The risk score or level and associated data can be encrypted to ensure protection to the individual when such data is accessed by healthcare providers, schools, employers, or the like. The tracing components of the system can digitally integrate and correspond with a variety of requestors to provide predetermined amount of information regarding the risk level of the individual. For example, such information can be only safe or not safe, or can provide details of the risk level.

The system discussed herein can be integral to economic re-entry during and after biological crises. For example, federal and state officials can mandate all approved tests to be collected to a secure, centralized, cloud-based database. Federal and state officials can subsequently require all enterprises and employees to be tested. The system can collect and log tests in the centralized database in a uniform format for analysis. The system can digitally certify individuals for re-entry based on CDC and medical guidelines in an updated manner. Results and certifications can be securely submitted electronically to, e.g., student rosters, employee lists, EMRs, Department of Motor Vehicles (DMVs), or the like. Certification results and checkpoints can be integrated throughout the social ecosystems to determine whether individuals can participate in certain events. Economic activity can thereby return to normal with less-restrictive, yet safe guidelines.

The system can be integrated with operations management and public communications systems. For example, the system can include a proprietary set of assets intended to support the development and adoption of the private system/public dashboards. A customized operations management dashboard can be used across the network of professional workforce, distributions, and industries. Public dashboard and communication systems can integrate with private management systems and inspire/power analytics platforms for tracking and tracing of data for individuals. Real-time analytics of tracing for any connections of positive case individuals, transit systems, events, or the like, can be provided in a dashboard user interface, with alert text and updates provided to individuals or entities via the dashboard. The dashboard can provide tools to visualize and narrow positive tests to specific areas (e.g., specific areas of a venue for an event), and based on location, industry and/or demographics. The dashboard can also provide focused notifications and alerts to specific sets of individuals and groups, and provides the ability to narrow the scope of the spread of high-risk individuals.

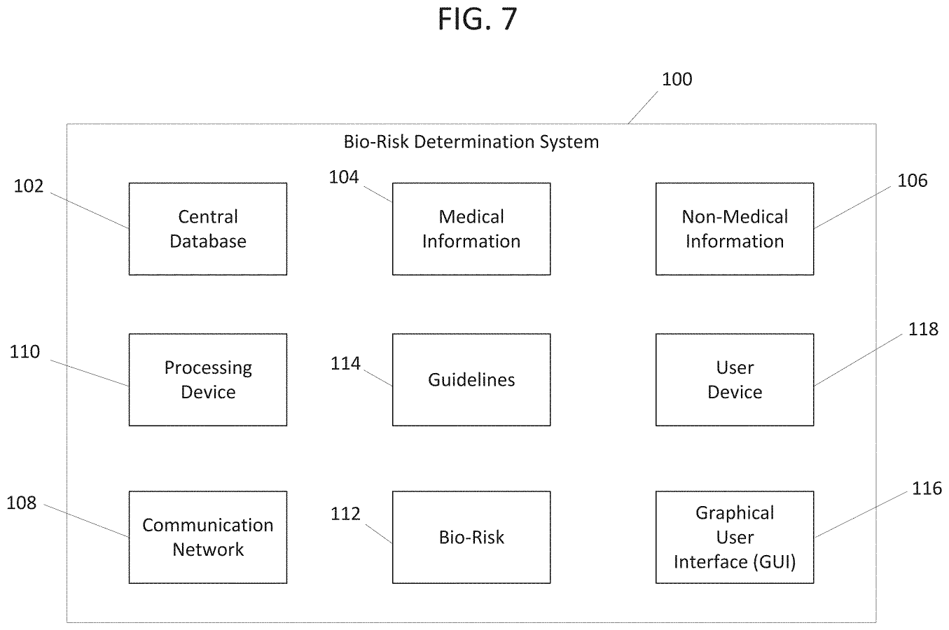

Additional detail of the present disclosure is described below in connection with FIGS. 7-19. Non-medical information may affect the bio-risk associated with the person. Such non-medical information can include, e.g., the person's surroundings including location and/or type of residence, manner of commute and/or route, job type, whether the commute passes through a high risk area, whether the commute involves others who are high risk, potential impact from weather, etc. Such non-medical information can be helpful on its own or in combination with point-of-care health data to determine the bio-risk associated with the person. Currently, there is no acceptable system that can communicate in real-time with different medical and/or non-medical systems to determine whether a particular individual may participate in an activity, transaction, or attendance at a particular location. Whether during a pandemic or as vaccinations become more widespread and businesses/venues begin to open, the present disclosure provides a real-time system that can be utilized to electronically certify that a person is approved to engage in an activity and/or to be present at a location using medical and/or non-medical data to determine a bio-risk of the person.

FIG. 7 is a diagram illustrating hardware and software components of the bio-risk determination system 100 (hereinafter “system 100”) of the present disclosure. The system 100 can be used in sports venues, music venues, public places, restaurants, stores, transit (e.g., airlines, buses, taxies, or the like), and a variety of other instances where a bio-risk of the individual is helpful in determining if the individual is safe for participating or entering into an area. With respect to the airline industry, one or more portions of the system 100 can be used before or during ticketing, before or during passage through security, before or during boarding, combinations thereof, or the like.

The system 100 includes a central database 102 that receives, in real-time, both medical information 104 and non-medical information 106 relating to individuals via a communications network 108. The medical information 104 can be any point-of-care data, as discussed above with respect to system 10. In some embodiments, the medical information 104 can include test results related to COVID-19, or any other virus, whether the individual has been vaccinated, or the like. In some embodiments, based on the medical information 104 input into the system 100, a processing device 110 of the system 100 can generate an electronic map to determine “hot spots” associated with an exposure to a particular virus or other exposure. In some embodiments, the “hot spots” map can be generated externally of the system 100 (e.g., based on publicly available medical information, private medical information collected, government information, medical industry information, combinations thereof, or the like), and the system 100 can receive as input the externally generated “hot spots” map to assist with implementation of the system 100. For example, based on COVID-19 test results collected as medical information 104, a map with “hot spots” can be generated in real-time, e.g., within a city, that represent high or elevated risk areas in the city. The generated map can be neighborhood specific to allow the system 100 to determine whether an individual's residence or commute through a particular area of the city increases the determined bio-risk 112 of the individual. In some embodiments, the generated map can be based on zip codes. In some embodiments, the generated map can be predictive by executing simulations to determine if non-hot spot areas are likely to become hot spots based on data trends. The determination of “hot spots,” and the initial determination of the medical test results, can be based on guidelines 114, e.g., CDC and/or medical guidelines on thresholds for point-of-care data to evaluate the biological illness level or risk, government guidelines, industry guidelines, combinations thereof, or the like. The guidelines 114 can be dynamically updated and/or reconfigured in real-time (or substantially real-time) to ensure that the resulting bio-risk 112 determination is accurately updated to reflect the most current standards. Accordingly, the system 100 utilizes data about the an individual and related to the individual, such as the residential address of an individual, where the individual shops or spends time, the individual's commute, etc., to determine the bio-risk 112 of the individual. The system can then utilize information relevant to the purpose of an individual at a location. For example, at a sport venue, an individual that is a spectator may have an entirely different risk profile from a sports player, a media representative or a vendor.

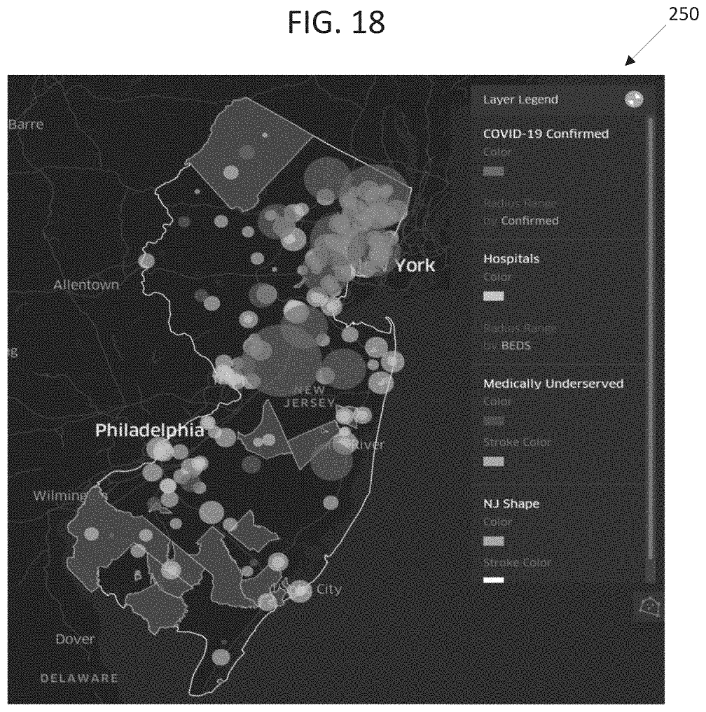

FIG. 18 is a screenshot of an example “hot spots” map 250 generated by the system 100 for the State of New Jersey. The map 250 provides a real-time, dynamic visual of COVID-19 cases, as well as hospital beds and medically underserved regions (as defined by a homeland security dataset). Although shown as a statewide map, it should be understood that the user interface can be used to zoom in on the map to view regions of the state, specific cities of the state, or detailed areas of the cities. As an example, zooming in on Trenton, N.J. would provide an indication of the COVID-19 “hot spots” in the city. Such information can be used to determine the public transportation passing through the respective “hot spots” and/or whether individuals reside within the “hot spots” to weigh the bio-risk determination for the individual. The “hot spots” or “hot zones” within the dynamic map can be tracked in real time (or substantially real time) to accurately understand the effect of such high or elevated risk areas on the commute or residence of individuals. In some embodiments, the system can keep track of exposure of individuals based on the hot zone analysis, including the name, last test date and result, days since last test, testing city, testing site, employee ID, and zip code. Such information can be used in combination with the “hot spots” map to determine the bio-risk status of the individual. Benchmarks can also be output by the system to compare the bio-risk or health status of individuals of one area relative to other areas (e.g., a specific city as compared to state values).

The non-medical information 106 can include, e.g., demographics, type of profession, residential location, job location, type of commute, public transportation, travel information, individual type, type of residential location (house or apartment), roommates and their medical and/or non-medical information, whether the commute passes through high risk areas, whether the commute involves others from high risk areas, potential impact from weather, combinations thereof, or the like. As an example, commuting via subway can be considered more high risk than driving alone in a car. However, if the individual commutes in a car with other individuals who live or conduct activities in high risk areas, commuting in the car with such individuals may also be considered high risk. Profession can include the type of job performed by the individual. As an example, if the individual performs a socially distanced job, such individual's bio-risk may be lower than an individual who works in the medical profession with COVID-19 patients. Residential location and job location can be used in combination with the “hot spots” map generated by the system 100 to determine if the individual lives in or commutes to a high-risk area (e.g., high-risk environment). Commute can include information on how individuals commute between their residential location and their job location, and the individuals involved in the commute. For example, commute information can include whether the individual drives in their own vehicle to work (e.g., resulting in a socially distanced and lower risk commute), whether the individual participates in carpooling (e.g., taking into account the bio-risk of individuals sharing the vehicle with the individual), whether the individual takes public transportation (e.g., taking into account if the individual walks through or takes certain types of public transportation that pass through high-risk areas or hot spots), combinations thereof, or the like. The non-medical information 106 can include not only commute information, but also travel of the individual outside of the commute, such as travel to a grocery store or for other errands.

The public transportation information can include the different types of public transportation available within a city and/or options for travel into the city. For example, the system 100 can include information that is specific to each bus, subway and/or train line to determine if the route of these modes of transport passes through “hot spots” associated with a virus. The system 100 can therefore determine if a subway line starting in the Bronx and ending in Brooklyn passed through specific “hot spots” along its route, and whether an individual travelling from point A to point B, included travel through these “hot spots,” results in an increase in the bio-risk of the individual. The system 100 can similarly determine if a train arriving in New York City from Washington, D.C. passed through cities that may be considered “hot spots,” and if an individual traveling this route is considered to have a higher bio-risk based on such travel through the “hot spots.” The system 100 can similarly determine if an individual arriving by airplane to New York City started their travel in an origin that is considered a “hot spot,” or if the individual had a layover in a city considered to be a “hot spot.” As noted above, the “hot spot” map can be updated in real time (or substantially in real time) to ensure an accurate determination of how an individual's commute or an individual's activities outside of the commute may affect the bio-risk of the individual.

Travel information can be based on a questionnaire provided to the individual to determine if the individual has traveled in a predetermined period of time (e.g., past two weeks). The individual type can affect the type and/or amount of information needed to determine the bio-risk of an individual. For example, if the individual type is an employee of a venue who works in a socially distanced area of the venue, the amount of information needed to determine the bio-risk of such individual may be reduced. As a further example, if the individual type is an employee of a venue who works directly with individuals entering and existing the venue, the amount of information needed to determine the bio-risk of such individual may be increased. As a further example, if the individual type is an attendee of an event at the venue (e.g., a fan), due to the interaction of the individual with other fans, the amount of information needed to determine the bio-risk of such individual may be increased.

With respect to travel information, in some embodiments, the individual can be asked to scan in or input a photograph of an identification card, driver's license, or passport as part of the registration process. In some embodiments, such input can automatically associate travel plans of the individual within the system 100. In some embodiments, the system 100 can be used to capture a photograph of the face of the individual and/or the iris, and such information can be subsequently used for biometric authentication of the individual for access to the system 100 using facial and/or iris recognition technology. In some embodiments, post or during registration, scanning of the identification card, driver's license or passport can be used to verify the identity of the individual to ensure a secure process.

The processing device 110 receives as input the medical information 104, the non-medical information 106, and the guidelines 114, and can determine the bio-risk 112 associated with each individual. The system 100 can include a graphical user interface (GUI) 116 to allow for viewing and/or input of data into the system 100. The GUI 116 can be used by administrators to ensure operate of the system 100 and to input information into the system 100 as needed. The system 100 can be in communication via the communication network 108 with a plurality of user devices 118 (e.g., mobile devices, smart phones, or the like). The system 100 can thereby provide a bio-risk certification or report in an electronic manner to the user device 118. In some instances, the GUI 116 can be located on the user device 118.

The GUI 116 can be used to initially register the individual with the system 100. The registration information can include basic information, such as the name, address, phone number, birthday, and e-mail address of the individual, as well as more detailed information, such as the non-medical and medical information discussed above. During or post-registration, the individual can input into the system using the GUI 116 a future event or activity that the individual will be participating in. Based on date (and optionally time) of the event, the system 100 can necessitate one or more actions by the individual within a predetermined threshold of the event/activity date to determine the bio-risk of the individual

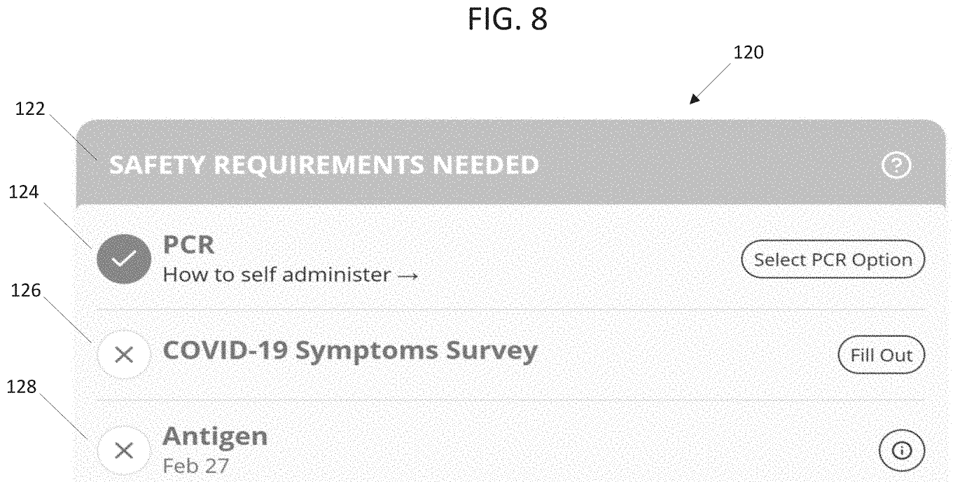

FIGS. 8-17 are sample screen shots of a user interface illustrating examples of implementation of the system 100. However, it should be understood that the system 100 and user interface for input and output of data to the user can be dynamically reconfigurable in a flexible manner to achieve the desired implementation of the user. The medical and/or non-medical data used by the system 100 can be selected as needed depending on the specific scenario(s) and desired implementation of the user. After the individual has met the requirements of the system 100, the system 100 can generate a bio-risk in the form of an electronic report that indicates whether the individual's bio-risk is sufficiently low or acceptable to attend the event or activity. In addition to visual cues (e.g., text, color coding, or the like) to indicate whether the individual's bio-risk level is sufficiently low to attend the event or activity, the electronic report can include a scannable code (e.g., a barcode, a QR code, or the like) that can be scanned at the venue of the event or activity.

The system 100 can involve a multi-step clearance process that includes specific input into the system 100 within a predetermined or preselected threshold period of time from the date of the event or activity. As an example, FIG. 8 is a sample screen shot of a user interface illustrating an example of an electronic certification report 120 capable of being generated by the system 100. The report 120 illustrates the interface visible to the individual who will be attending an event or activity. The report 120 can include a status section 122 that indicates whether the individual has meet all requirements for the bio-risk determination, and the safe or not safe status of the individual based on bio-risk determination. The status section 122 can be color coded, e.g., red to indicate no requirements met, yellow to indicate some requirements met, green to indicate all requirements met, or the like.

The system 100 can be reconfigured to include one or more different types of input for determination of the bio-risk of the individual. As an example, FIG. 8 shows that the individual is required to take a polymerase chain reaction (PCR) test 124, a symptoms survey 126, and an antigen test 128. The PCR test 124 can be taken within a predetermined period (e.g., temporal period) of time of the event or activity. For example, the PCR test 124 can be required within 72 hours of the event or activity. However, it should be understood that the threshold for the PCR test 124 can be selected by, e.g., the organization hosting the event or activity. The symptoms survey 126 can be an electronic questionnaire performed by the individual before arriving to the location of the event or activity. The antigen test 128 can be a rapid antigen test performed at the facility hosting the event or activity. For example, the individual can arrive to the facility before the event or activity, the antigen test 128 can be performed, and the individual can be informed on the user device when the antigen test 128 result has been received. In some embodiments, the facility hosting the event or activity can include a laboratory to expedite the antigen test 128 results. In some embodiments, a rapid antigen test can be performed with results available in about 5-8 minutes from testing. In some embodiments, the results of the antigen test can be available within 45 minutes from testing. In some embodiments, the antigen test can be sent to a remote facility from the facility hosting the event or activity. In some embodiments, the antigen test can be performed within 24 hours of the event or activity.

In some embodiments, based on the temporal period requirements of each of the responses or inputs into the system 100 for determining the bio-risk, some responses are collected at the facility hosting the event, while other responses can be collected remote from the facility hosting the event. For example, the PCR test 124 can be conducted at a medical facility remote from the facility hosting the event, or can be performed by the individual themselves and uploaded as input to the system 100. As a further example, the questionnaire associate with the symptoms survey 126 can be completed while the individual is remote from the facility hosting the event. The antigen test 128 can be conducted at the facility hosting the event, e.g., a laboratory located at the facility, to ensure the most accurate and recent antigen test results are used in the bio-risk determination.

The report 120 generally displays the safety requirements required by one type of facility. The report 120 provides an electronic, real-time interface or dashboard for individuals (e.g., fans, employees, or the like) to obtain permission to enter a facility on a specific date and time. It should be understood that different types of safety requirements may be needed for different facilities, and/or for different individuals. For example, a fan entering a facility may need all three of the listed features, while an employee may only need two out of three of the listed features, and media may only need one out of three of the listed features.

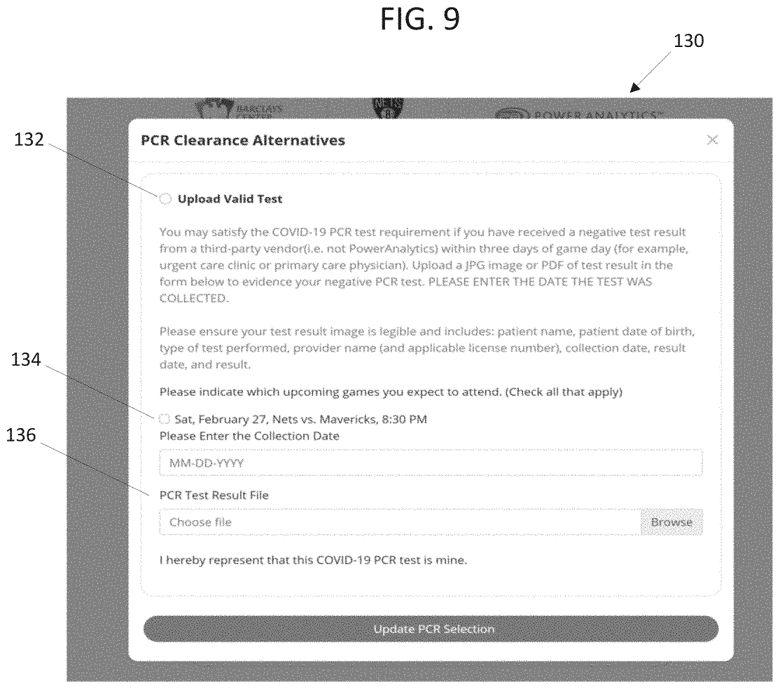

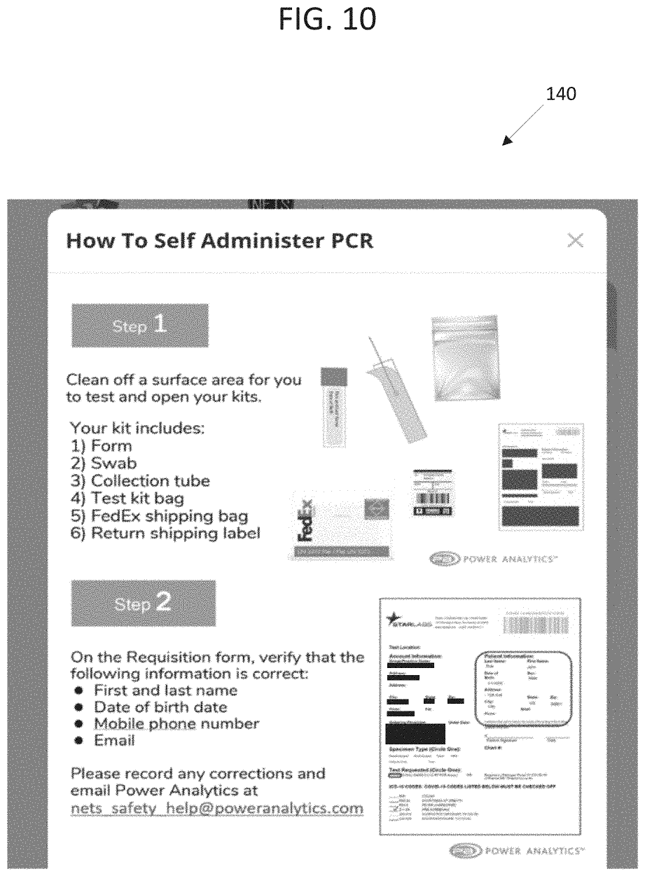

FIG. 9 is a sample screen shot of a user interface illustrating an example of a PCR test input 130 generated by the system 100. The input 130 can include a section 132 for uploading a valid test result, e.g., a test result from a third-party medical facility. In some embodiments, the PCR test results can be uploaded, ordered by mail, or provided in alternative ways to the system 100. The input 130 can include a section 134 indicating the date and time of the event or activity the individual is seeking to participate in. The input 130 can include one or more sections 136 for input of the PCR administration date and upload of the PCR test result file for input into the system 100. In some embodiments, a PCR test can be administered by the individual. In such embodiments, PCR administration instructions 140 can be provided to the user, as illustrated by FIG. 10. The PCR test results can be input to the system 100 automatically after the test analysis has been completed. The individual can be notified via, e.g., a mobile device, that the PCR test result has been updated. The individual can access the user interface associated with the system 100 to obtain details about the PCR test result.

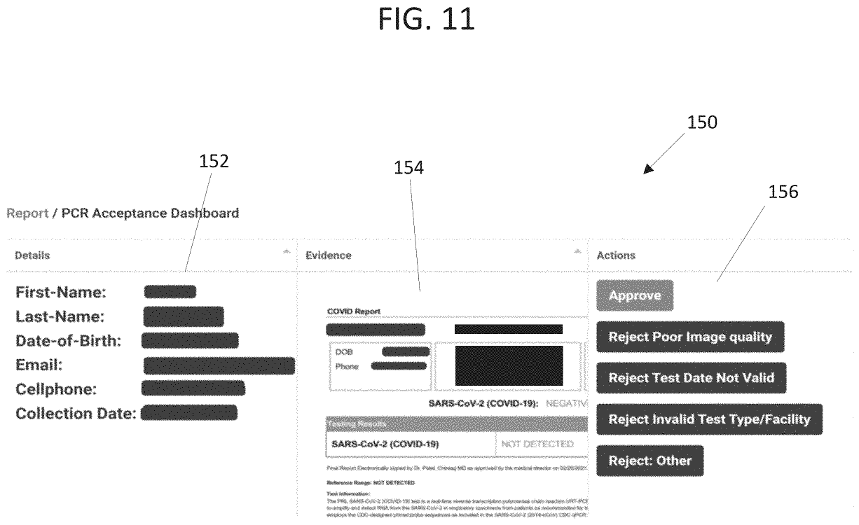

FIG. 11 is a sample screen shot of a user interface illustrating an example of a PCR acceptance dashboard 150 capable of being generated by the system 100. The dashboard 150 can be visible to administrators of the system 100 on the back end, and may not be visible to the individual being tested. However, the individual would be capable of seeing a visual indication of the results of the PCR test on, e.g., the report 120 of FIG. 8. Such indication on the report 120 can be updated substantially in real-time when the PCR test results have been obtained. As shown in FIG. 11, the dashboard 150 can include general information on the individual in a details section 152, such as the name, birthday, e-mail address, telephone number, and test collection data. The dashboard 150 can include an evidence section 154 with information on the type of test administered, the doctor or medical facility involved, and the test result. The dashboard 150 can include an actions section 156 with visual cues of the results of the test, such as approve, reject poor image quality, reject test date not valid, reject invalid test type/facility, reject other, or the like. The dashboard 150 can communicate the results of the PCR test to the system 100, and updates the individual's certification report 120 in real-time to notify the individual if one of the requested steps has been completed. The dashboard 150 can provide a convenient way of reviewing and accepting/denying a third-party PCR test uploaded to the system 100.

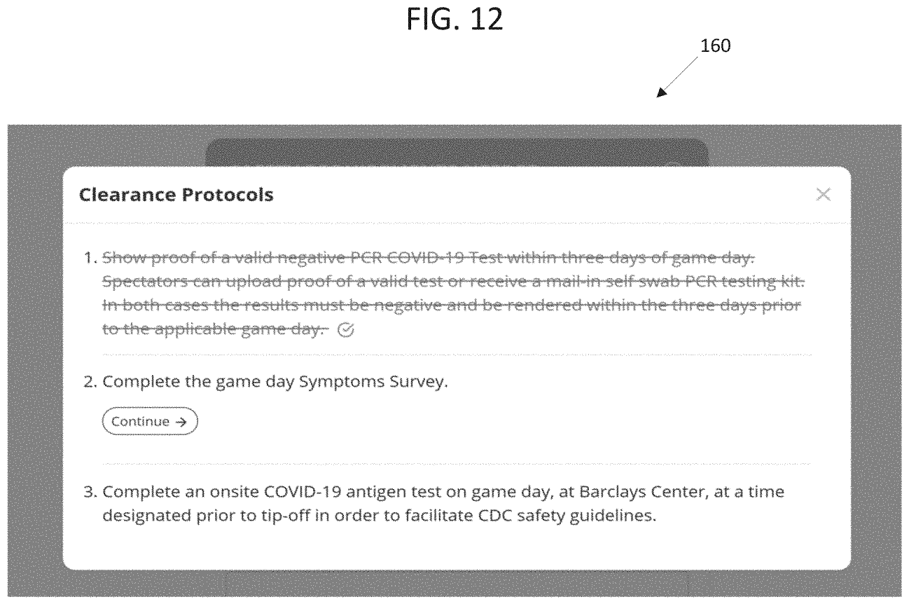

FIG. 12 is a sample screen shot of a user interface illustrating an example of a clearance protocols status 160 capable of being generated by the system 100. The status 160 can indicate in real-time which requirements the individual has completed prior to attending the event or activity. The status 160 can also indicate which requirements the individual is eligible to complete, e.g., by activating the “Continue” button for the symptoms survey when the current date/time is within the predetermined timeframe for completing the symptoms survey. As an example, as illustrated in FIG. 12, the status 160 indicates that the PCR test has been completed, and the remaining steps include the symptoms survey and the antigen test to be completed the day of the activity or event.

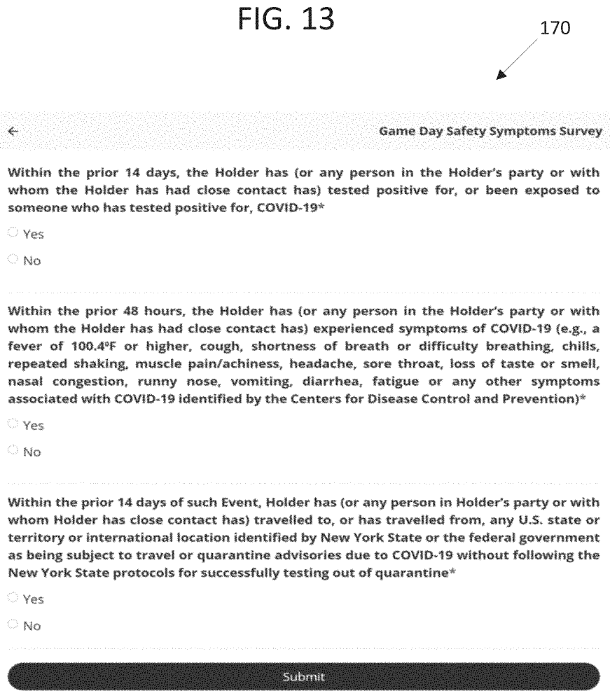

FIG. 13 is a sample screen shot of a user interface illustrating an example of a symptoms survey input 170 capable of being generated by the system 100. The input 170 can include any symptom questions desired by the facility hosting the event or activity and, in some embodiments, can include questions required by medical and/or government guidelines. For example, the symptoms survey can require input regarding contact with others who have tested positive for COVID-19, whether the individual has experienced symptoms of COVID-19 within the prior 48 hours, and whether the individual has been in close contact with individuals who have traveled or if the individual themselves has traveled to states having quarantine or travel advisories due to COVID-19. In some embodiments, the symptoms survey input 170 can include questions regarding the commute of the individual or the type of work the individual performs. As noted above, these non-medical questions can be used to ascertain if the individual participates in high risk activities that could increase the bio-risk of the individual.

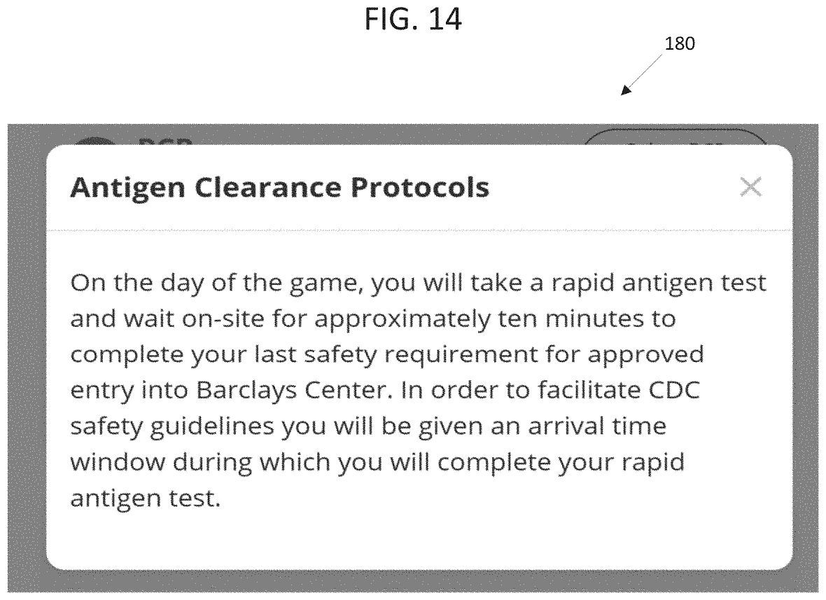

FIG. 14 is a sample screen shot of a user interface illustrating an example of an antigen clearance protocol 180 capable of being generated by the system 100. The protocol 180 can be reviewed by the individual in preparation for attending the activity or event. As noted in FIG. 14, in some embodiments, the protocol 180 can provide information to the individual regarding the how the antigen test will be performed on the day of the activity or event. The protocol 180 can prepare for the individual for attending the activity or event, ensuring that sufficient time is allotted for the test. Upon arrival to the facility, the antigen test can be performed and the user can be notified on their mobile device when the results have been completed. The results can be updated in real-time on the certification report of FIG. 8 to indicate if the individual's bio-risk is sufficiently low to allow participation in the activity or event.

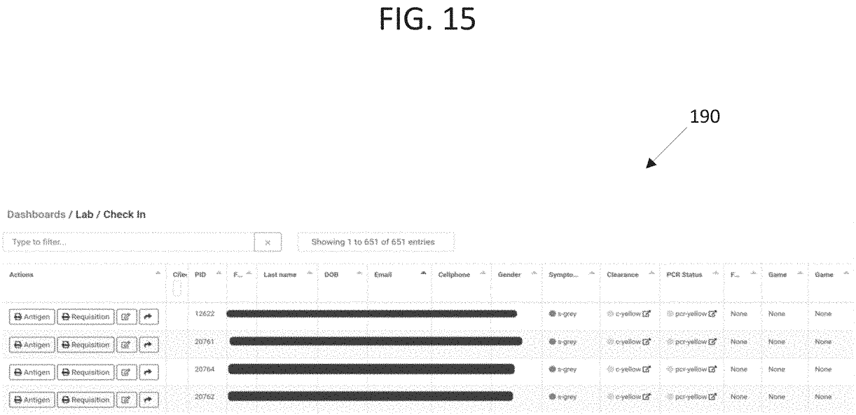

FIG. 15 is a sample screen shot of a user interface illustrating an example of a laboratory check-in dashboard 190 capable of being generated by the system 100 for performing the antigen test. The dashboard 190 can be used by administrators at the facility to schedule individuals for the antigen test, uploading antigen test results, and displaying the results of the PCR test and/or the symptoms survey. Each individual can be assigned a unique alphanumeric identification to maintain the record in the system 100. The dashboard 190 allows the administrator to determine if the individual has completed all prior steps and is ready for the antigen test. The dashboard 190 can be used to track the status in the process for different types of individuals, e.g., fans, employees, media, or the like, and provides a real-time status for each respective individual. As noted above, the system 100 is dynamically reconfigurable. Therefore, if guidelines change or if the facility decides to enact stricter guidelines (e.g., a PCR test within one day of the event), the database would be updated in real-time and each of the PCR tests in the dashboard 190 taken outside of a one day timeframe would turn orange or red to indicate action is needed to retake the PCR test. The system 100 thereby provides a real-time ability for reconfiguration of the system based on the needs of the facility.

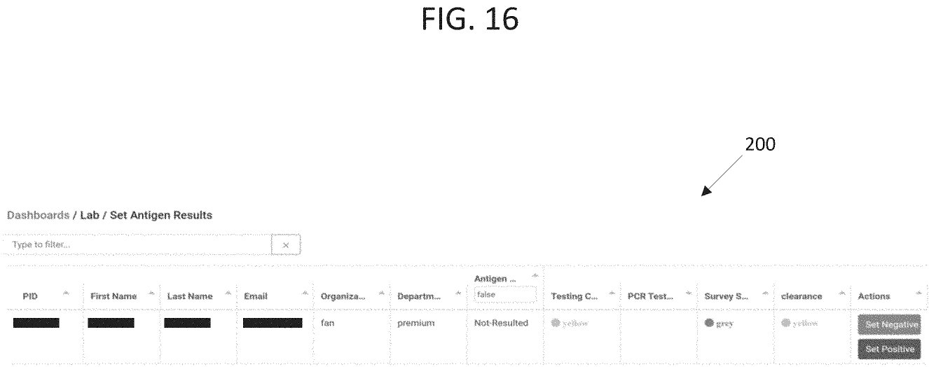

FIG. 16 is a sample screen shot of a user interface illustrating an example of an antigen results dashboard 200 capable of being generated by the system 100. The dashboard 200 can be used by administrators at the facility to input the results of the antigen test based on laboratory testing results. The dashboard 200 can include the unique alphanumeric identification of the individual, the PCR test result, and the symptom survey result. Once the test antigen test result is available, the administrator can manually select whether the result is negative or positive. In some embodiments, the laboratory equipment can be communicatively connected to the system 100 such that the antigen test results are automatically input into the system 100 upon determination of the antigen test result, thereby bypassing the administrator. Once input, the antigen test result can be updated in real-time in the certification report 120 of FIG. 8 and a notification can be electronically transmitted to the individual's mobile device indicating whether the individual can enter the facility for the event or activity.

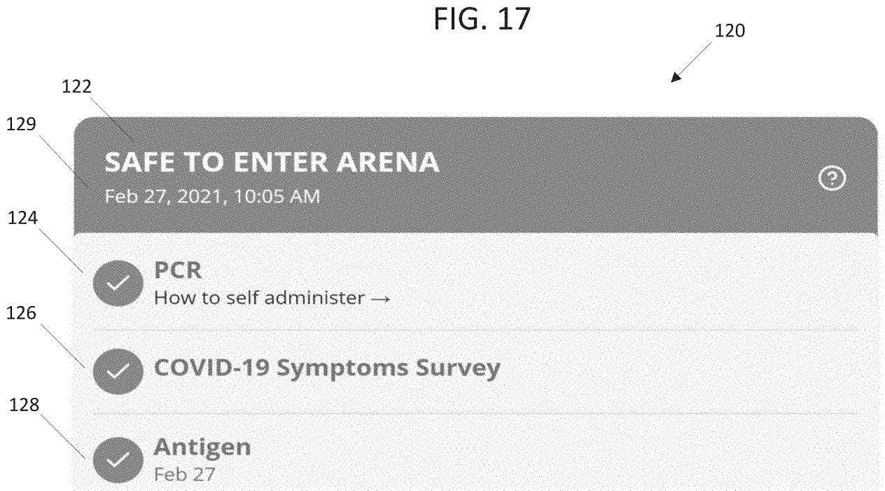

FIG. 17 is a sample screen shot of a user interface illustrating an example of the electronic certification report 120 (e.g., the bio-risk certification report) after each of the determination steps have been successfully completed. Rather than providing an in progress status, the status section 122 reflects a green color for convenient visibility that the individual is at a low bio-risk and it is safe for the individual to enter the facility. The PCR test 124, the symptoms survey 126 and the antigen test 128 sections each include a green checkmark to reflect successful passage of each of these steps. The antigen test 128 section includes the date on which the test was successfully taken. The status section 122 includes a date/time section 129 (e.g., a time stamp) that reflects the date/time on which each of the steps was completed by the individual and the date/time on which access to the facility was granted to the individual. The time stamp in the date/time section 129 can be programmed to expire or lapse after a predetermined period of time (e.g., 6 hours, or the like). Therefore, if an individual attempts to enter the facility beyond the 6 hour timeframe, the report 120 can be refreshed and would indicate that the individual has not met the requirements to enter at the requested time.

Although discussed herein as being used for determining if an individual can participate in an activity or event in a facility, it should be understood that the system 100 can be used for a variety of purposes. For example, the system 100 can be used to determine if more or less credit should be extended to an individual, or any other use where a decision must be made based on a bio-risk associated with the individual.

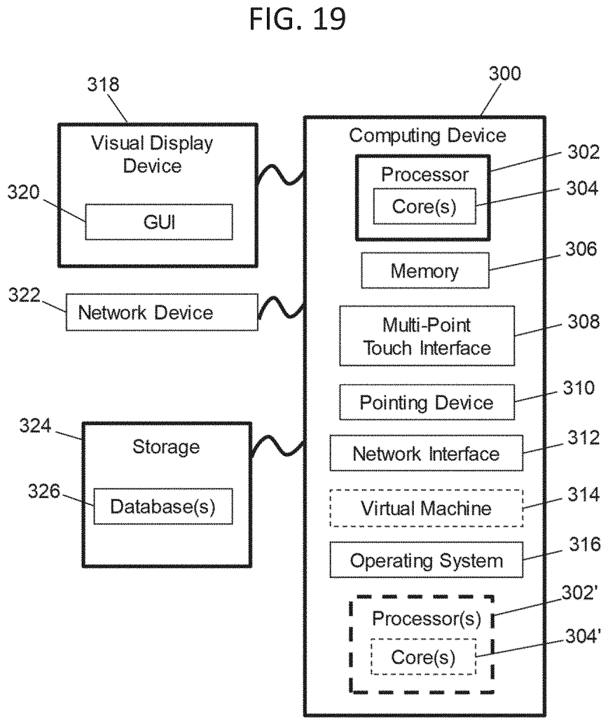

FIG. 19 is a diagram of a computing device 300 capable of being implemented with the system 100. The computing device 300 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more flash drives), and the like. For example, memory 306 included in the computing device 300 may store computer-readable and computer-executable instructions or software for implementing exemplary embodiments of the present disclosure (e.g., instructions for controlling the communication network or interface, the processing device, the user interface, combinations thereof, or the like). The computing device 300 also includes configurable and/or programmable processor 302 and associated core 304, and optionally, one or more additional configurable and/or programmable processor(s) 302′ and associated core(s) 304′ (for example, in the case of computer systems having multiple processors/cores), for executing computer-readable and computer-executable instructions or software stored in the memory 306 and other programs for controlling system hardware. Processor 302 and processor(s) 302′ may each be a single core processor or multiple core (304 and 304′) processor.

Virtualization may be employed in the computing device 300 so that infrastructure and resources in the computing device 300 may be shared dynamically. A virtual machine 314 may be provided to handle a process running on multiple processors so that the process appears to be using only one computing resource rather than multiple computing resources. Multiple virtual machines may also be used with one processor. Memory 306 may include a computer system memory or random access memory, such as DRAM, SRAM, EDO RAM, and the like. Memory 306 may include other types of memory as well, or combinations thereof.

A user may interact with the computing device 300 through a visual display device 318 (e.g., a personal computer, a mobile smart device, or the like), such as a computer monitor, which may display one or more user interfaces 320 (e.g., a GUI) that may be provided in accordance with exemplary embodiments. The computing device 300 may include other I/O devices for receiving input from a user, for example, a keyboard or any suitable multi-point touch interface 308, a pointing device 310 (e.g., a mouse), or the like. The keyboard 308 and the pointing device 310 may be coupled to the visual display device 318. The computing device 300 may include other suitable conventional I/O peripherals.

The computing device 300 may also include one or more storage devices 324, such as a hard-drive, CD-ROM, or other computer readable media, for storing data and computer-readable instructions and/or software that implement one or more portions of the system 100. Exemplary storage device 324 may also store one or more databases 326 for storing any suitable information required to implement exemplary embodiments. For example, exemplary storage device 324 can store one or more databases 326 for storing information, such as data relating to the medical information, the non-medical information, guidelines, or the like, and computer-readable instructions and/or software that implement exemplary embodiments described herein. The databases 326 may be updated by manually or automatically at any suitable time to add, delete, and/or update one or more items in the databases.

The computing device 300 can include a network interface 312 configured to interface via one or more network devices 322 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 312 may include a built-in network adapter, network interface card, PCMCIA network card, card bus network adapter, wireless network adapter, USB network adapter, modem or any other device suitable for interfacing the computing device 300 to any type of network capable of communication and performing the operations described herein. Moreover, the computing device 300 may be any computer system, such as a workstation, desktop computer, server, laptop, handheld computer, tablet computer (e.g., the iPad™ tablet computer), mobile computing or communication device (e.g., the iPhone™ communication device), or other form of computing or telecommunications device that is capable of communication and that has sufficient processor power and memory capacity to perform the operations described herein.

The computing device 300 may run an operating system 316, such as versions of the Microsoft® Windows® operating systems, the different releases of the Unix and Linux operating systems, versions of the MacOS® for Macintosh computers, embedded operating systems, real-time operating systems, open source operating systems, proprietary operating systems, or other operating systems capable of running on the computing device and performing the operations described herein. In exemplary embodiments, the operating system 316 may be run in native mode or emulated mode. In an exemplary embodiment, the operating system 316 may be run on one or more cloud machine instances.

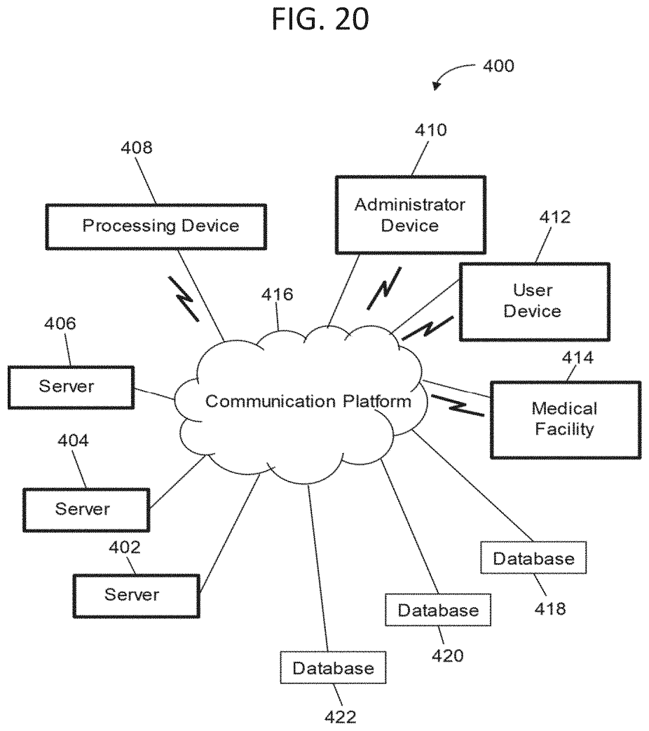

FIG. 20 is a diagram of an example bio-risk determination system environment 400. The environment 400 can include servers 402, 404, 406 operatively coupled to a processing device 408, administrative device 410, user device 412, and a medical facility 414, via a communication platform 416, which can be any network over which information can be transmitted between devices communicatively coupled to the network. For example, the communication platform 416 can be the Internet, Intranet, virtual private network (VPN), wide area network (WAN), local area network (LAN), and the like. In an embodiment, the communication platform 416 can be part of a cloud environment. The environment 400 can include repositories or databases 418, 420, 422, which can be operatively coupled to the servers 402, 404, 406, as well as to the processing device 408, the administrator device 410, the user device 412, and the medical facility 414, via the communications platform 416. In exemplary embodiments, the servers 402, 404, 406, processing device 408, administrator device 410, user device 412, medical facility 414, and databases 418, 420, 422 can be implemented as computing devices (e.g., computing device 300). Those skilled in the art will recognize that the databases 418, 420, 422 can be incorporated into one or more of the servers 402, 404, 406 such that one or more of the servers 402, 404, 406 can include databases 418, 420, 422. In an embodiment, the database 418 can store the medical information, the database 420 can store the non-medical information, and the database 422 can store the guidelines. In an embodiment, a single database 418, 420, 422 can store the medical information, the non-medical information, and the guidelines. In an embodiment, embodiments of the servers 402, 404, 406 can be configured to implement one or more portions of the system 100.

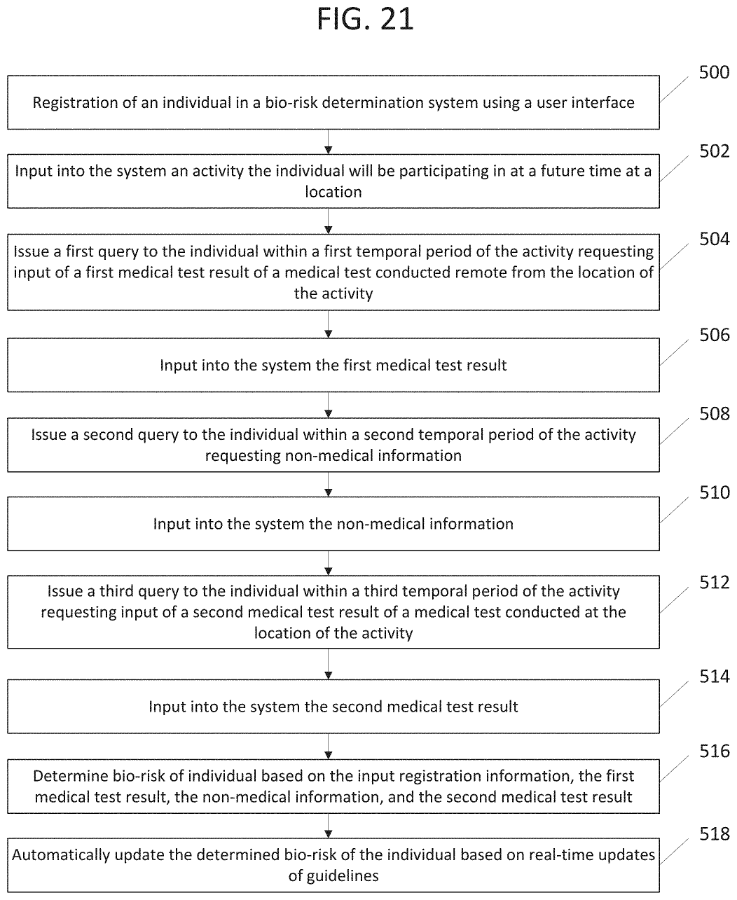

FIG. 21 is a flowchart illustrating an example process of implementing the system 100. At step 500, the individual can electronically register with the system using a user interface. The registration process can collect general information about the individual, such as the name, address, birthdate, and contact information. The registration process can also include information about the job and/or job type of the individual, the type of commute typically performed by the individual, or the like. Such non-medical information can be used in determining if the individuals profession and/or commute affects the bio-risk to be determined for the individual.

At step 502, the individual can input into the system an activity the individual will be participating in at a future time at a specific location. The activity can be any type of public activity, e.g., a sports event, a music/concert event, a movie a movie theater, a museum exhibit, airline travel, public transportation travel, or the like. It should be understood the activities provided herein are merely intended to provide a non-limiting example of the types of activities. At step 504, the system can issue a first electronic query to the individual within a first temporal period of the activity requesting input of a first medical test result of a medical test conducted remote from the location of the activity. The first temporal period can be, e.g., three days before the scheduled activity. The method test result can be, e.g., the PCR test result discussed herein. The individual can undergo the PCR test remote from the location of the activity, with the test result being input into the system automatically or substantially automatically at step 506. The system can internally confirm that the PCR test has been accepted and the first electronic query is satisfied.

At step 508, the system can issue a second electronic query to the individual within a second temporal period of the activity requesting input of non-medical information. The second temporal period is shorter than the first temporal period, e.g., two days or one day before the scheduled activity. The non-medical information can be, e.g., the questionnaire discussed herein, information regarding the individual's activities that may affect the health of the individual, or the like. In some embodiments, at step 508, the system can request input of medical information in the form of general inquiries (e.g., not medical test results), such as the individual's symptoms. At step 510, the user electronically inputs into the system the requested non-medical information. The system can internally confirm that the non-medical information provided is acceptable to satisfy the second electronic query.

At step 512, the system can issue a third electronic query to the individual within a third temporal period of the activity requesting input of a second medical test result of a medical test conducted at the location of the activity. The third temporal period is shorter than the first and second temporal periods, e.g., the day of the activity, an hour before the activity begins, or the like. In some embodiments, the medical test can be a COVID-19 antibody test to be conducted at the facility hosting the activity. For example, the individual can arrive to the facility within a predetermined timeframe (e.g., an hour before the activity begins), the medical test can be performed, and the results can be available before activity begins. At step 514, the medical facility or laboratory located at the facility hosting the event can input the medical test results automatically into the system once the test results are available. The system can internally review the input data to confirm that the second medical test result satisfies the requirements of the third query, and to confirm that each of the three queries has been satisfies such that the individual can participate in the activity.

At step 516, based on the input information, the system determines the bio-risk of the individual. The system takes into account the information provided during registration, as well as the subsequent medical test results and non-medical information, to determine the bio-risk associated with the individual. At step 518, the system can automatically update the determined bio-risk of the individual based on real-time updates of medical, local and/or government guidelines. In some embodiments, the system can be automatically updated and/or reconfigured based on changes in guidelines to adjust parameters of the system, such as the temporal periods associated with each of the queries, values associated with the medical test results, or the like.

The system 100 thereby provides an efficient and convenient means for determining the bio-risk of an individual based on non-medical information (and optionally in combination with medical information). The system 100 can be used to determine if the environment surrounding the individual during their day-to-day activities, or during their commute to the workplace or facility, places the individual at a higher bio-risk than is desired for entrance to a particular facility. The determination can be performed in real-time to ensure accurate and timely results are provided to the end user.

Having thus described the system and method in detail, it is to be understood that the foregoing description is not intended to limit the spirit or scope thereof. It will be understood that the embodiments of the present disclosure described herein are merely exemplary and that a person skilled in the art can make any variations and modification without departing from the spirit and scope of the disclosure. All such variations and modifications, including those discussed above, are intended to be included within the scope of the disclosure.

What is claimed is:

1. A bio-risk determination system, comprising:
a database including information on an individual, the database configured to receive data relating to an activity the individual is to participate in at a future time at a location; and
a processing device configured to:
receive a first input at a user interface of a mobile electronic device from the individual in response to a first query during a first temporal period prior to the activity;
receive a second input at the user interface from the individual in response to a second query during a second temporal period prior to the activity, the second temporal period is different from the first temporal period;
analyze the first and second inputs, and generate a bio-risk of the individual based on the first and second inputs in real-time; and
display a report in the user interface of a mobile electronic device, the report indicating the bio-risk generated for the individual.

2. The bio-risk determination system of claim 1, wherein the activity is at least one of a sports event, a musical event, airline travel, bus travel, or train travel.

3. The bio-risk determination system of claim 1, wherein the first query is a request for a first medical test result associated with the individual.

4. The bio-risk determination system of claim 3, wherein the first medical test result is based on a polymerase chain reaction (PCR) test.

5. The bio-risk determination system of claim 3, wherein the first medical test result is based on a first medical test conducted remotely from the location of the activity.

6. The bio-risk determination system of claim 5, wherein the first medical test result is automatically input in real-time into the database from a medical facility conducting the first medical test.

7. The bio-risk determination system of claim 1, wherein the second query is an electronic questionnaire regarding symptoms of the individual.

8. The bio-risk determination system of claim 1, wherein the processing device is configured to receive a third input at a user interface from the individual in response to a third query during a third temporal period prior to the activity.

9. The bio-risk determination system of claim 8, wherein the third query is a request for a second medical test result associated with the individual.

10. The bio-risk determination system of claim 9, wherein the second medical test result is based on a COVID-19 antibody test.

11. The bio-risk determination system of claim 9, wherein the second medical test result is based on a second medical test conducted at the location of the activity.

12. The bio-risk determination system of claim 8, wherein the third temporal period is different from the first and second temporal periods.

13. The bio-risk determination system of claim 8, wherein the first temporal period is a longer time period from the activity than the second and third temporal periods, and the second temporal period is a longer time period from the activity than the third temporal period.

14. The bio-risk determination system of claim 11, wherein the second medical test is conducted on the day on which the individual is participating in the activity.

15. The bio-risk determination system of claim 8, wherein the processing device is configured to update the bio-risk of the individual based on the third input in real-time.

16. The bio-risk determination system of claim 1, wherein the report includes a code capable of being scanned to display information associated with the report on another user interface of an electronic device, and wherein the code is a QR code or a barcode.

17. The bio-risk determination system of claim 1, wherein the processing device is configured to automatically update the bio-risk of the individual based on updates in medical or government guidelines or thresholds regarding a biological illness level or risk.

18. The bio-risk determination system of claim 1, wherein the database is configured to receive as input and electronically store data relating to an environment associated with the individual.

19. The bio-risk determination system of claim 18, wherein the processing device is configured to generate a dynamic hot spot map based on the data relating to the environment associated with the individual.

20. The bio-risk determination system of claim 19, wherein the processing device is configured to automatically update the bio-risk associated with the individual based on travel of the individual through hot spots of the dynamic hot spot map.

21. A method of determining a bio-risk, the method comprising:
receiving at a database information on an individual;
receiving at the database data relating to an activity the individual is to participate in at a future time at a location;
receiving a first input at a user interface of a mobile electronic device from the individual in response to a first query during a first temporal period prior to the activity;
receiving a second input at the user interface from the individual in response to a second query during a second temporal period prior to the activity, the second temporal period is different from the first temporal period;
analyzing the first and second inputs with a processing device, and generating a bio-risk of the individual based on the first and second inputs in real-time; and
displaying a report in the user interface, the report indicating the bio-risk generated for the individual.

22. A non-transitory computer-readable medium storing instructions at least for generating a bio-risk that are executable by a processing device, wherein execution of the instructions by the processing device causes the processing device to:
receive at a database information on an individual;
receive at the database data relating to an activity the individual is to participate in at a future time at a location;
receive a first input at a user interface of a mobile electronic device from the individual in response to a first query during a first temporal period prior to the activity;
receive a second input at the user interface from the individual in response to a second query during a second temporal period prior to the activity, the second temporal period is different from the first temporal period;
analyze the first and second inputs, and generating a bio-risk of the individual based on the first and second inputs in real-time; and
display a report in the user interface, the report indicating the bio-risk generated for the individual.

23. A bio-risk system for public transportation, comprising:
a database including information on a plurality of individuals, the database configured to receive data relating to (i) travel information associated with each of the plurality of individuals, and (ii) at least one medical test result associated with each of the plurality of individuals;
a processing device configured to:
analyze the travel information and the at least one medical test result, and generate an electronic report;
transmit the electronic report to a mobile electronic device of at least one of the plurality of individuals; and
display the report with a user interface of the mobile electronic device in the form of a real-time health certification, the report including at least one of the medical test result, or an indication of a positive or negative result of the medical test result.

24. The bio-risk system of claim 23, wherein the at least one medical test result is a COVID-19 antibody test result.

25. The bio-risk system of claim 23, wherein the travel information is a travel itinerary or ticket.
